(19) United States
(12) Patent Application Publication
(10) Pub. No.: US 2008/0228174 A1
(43) Pub. Date: Sep. 18, 2008

(54) Drain Tube Assembly for Draining a Body Cavity

(76) Inventors: Kamal Ibrahim, London (GB); Andrew R. May, London (GB); John M. Elam, London (GB)

Correspondence Address: John L. Cordani;Carmody & Torrance, 50 Leavenworth Street, P.O. Box 1110, Waterbury, CT, 06721-1110, US

(21) Appl. No.: 11/795,936

(22) PCT Filed: Jan. 27, 2006
(86) PCT No.: PCT/GB06/00285

§ 371 Date: Mar. 10, 2008

(30) Foreign Application Priority Data
Jan. 27, 2005 (GB) 0501750.4
Jul. 14, 2005 (GB) 0514412.6

(51) Int. Cl. A61M 27/00

(52) U.S. Cl. 604/541

(57) ABSTRACT

A drain tube assembly for draining a body cavity, the assembly comprising a flexible drain tube having a distal end for insertion into the body cavity and a proximal end, the distal end having at least one drainage hole therein, and at least one attachment ring so as to provide an attachment for suture, the attachment ring being connected to the drain tube by a flexible arm permitting rotational movement of the attachment ring relative to the drain tube.

The present invention relates to a drain tube assembly for draining a body cavity, and in particular to a chest drain for use in thoracic surgery. The present invention also relates to a kit of parts for such a drain tube assembly. The present invention also relates to an anchoring device for a chest drain. The present invention further relates to a method of inserting a chest drain.

In thoracic surgery it is sometimes necessary to insert a tube, known in the art as a chest drain, into the chest cavity of a patient. Chest drain insertion is typically used to provide drainage of plural effusion and/or pneumothorax. It may be inserted in an emergency, or used post-operatively.

Various techniques are practiced by clinical personnel and the success of the securing of the chest drain is largely operator dependent. The best practice currently recommended in the UK is described in the British Thoracic Society Guidelines comprising a paper entitled “BTS [British Thoracic Society] Guidelines for the Insertion of a Chest Drain”, published in Thorax 2003; 58 (Suppl. II): ii 53-ii 59. Once a chest drain has been inserted into the chest cavity of a patient, the chest drain should ideally remain in place until it is clinically indicated to remove it. In the BTS paper there is a discussion of the best practice on how a chest drain should be secured in place after insertion into the chest cavity of a patient. The securing requires the use of suturing and self-adhesive dressing tape. The paper discloses in paragraph 13.3.4 and in FIG. 4 the use of a suture for securing the exterior surface of the chest drain to the skin of the patient. The chest tube is cylindrical, and the stay suture is wound tightly around the chest tube and then stitched to the skin and subcutaneous tissue. Self-adhesive wound dressing tape is wrapped around the chest tube over the wound suture to hold the suture in place on the tube and to secure a wound dressing over the wound site. An omental tag of the tape may be provided to allow the tube to lie a little away from the chest wall to prevent the tube kinking and tension at the insertion site.

The drain needs to be secured so as to be able to be held in place for a significant period. In thoracic surgery, for example, sometimes chest drains are inserted for only a few days, for example from 2 to 7 days, but occasionally a chest drain is inserted for up to a month, and the patient may either remain in hospital or be permitted to go home with the chest drain still inserted.

Unfortunately, a number of complications can arise during or after drain insertion. Such complications include drains falling out, the drain requiring re-insertion, the drain leaking, the insertion site becoming infected, or surgical emphysema. In a retrospective study carried out by one of the present inventors, on intercostal chest drains and their complications, the case notes of patients with pneumothorax were reviewed retrospectively to identify patients who need chest drain insertion, and patients who needed chest tube drainage of their plural effusion. In that retrospective study of 68 patients, the complications rate was 31%, of which 24% complications resulted from the drains having fallen out. The complications rate tended to depend on whether the chest drain had been inserted in a respiratory (i.e. specialist) ward as compared to any other ward, and whether the procedure was performed by a more experienced operator.

The aforesaid present inventor believes that although the British Thoracic Society has proposed guidelines for the insertion of a chest drain, these may not be generally practiced and medical personnel require additional training for securing chest drains in a reliable manner. The use of suturing and a lot of adhesive tape and gauze which is current practice is complicated, difficult to teach, and takes some time to master. Therefore the current practice to secure chest drains onto chest walls largely depends on the experience of an operator. However, chest drains are frequently placed by junior medical staff on the wards who receive no formal training or instructions especially when it comes to securing the chest drains. Furthermore, a recognition of any wound and gauze infection or leaking fluid from the wound would be delayed if the overlying taping was overly bulky. It could also be very uncomfortable for the patient (and doctor) when it comes to removing all the gauze and adhesive tape off the chest wall, especially if the drain needs to be re-inserted because it has fallen off. This is particularly the case in mobile patients on medical wards. Alternatively, it would also be uncomfortable for the patient if the chest drain had to be repositioned, or “pushed in a little” because it has slipped down or “pulled out a little” because it had been pushed too far into the chest cavity. Some patients need more than one chest drain, and this exacerbates the likelihood of complications. It is not uncommon practice for chest drains not to be re-inserted after they have fallen accidentally from the patient. This is frequently to avoid the discomfort and pain a patient may have to go through and hence a less than ideal drainage period must be accepted.

It is known for small bore tubes, in particular blood infusion tubes for connection to veins or arteries, for example a central line, to be attached to the skin of a patient by an anchoring attachment, for example a butterfly device well known to persons skilled in the art. Such devices are usable with such small tubes because the length of the lumen for insertion into the vein or artery is predetermined and also the tube is held, immediately external of the lumen, at a fixed orientation parallel to the skin. Thus the small bore tube is in a fixed orientation with regard to the skin of a patient and inserted to a predetermined depth.

Such anchoring systems cannot be employed for chest drains. This is because not only does the chest drain require a variable insertion length, but also the chest drain must also have a variable angle of insertion into the chest cavity of a patient. For example, the chest tube of the chest drain may be arranged either to point up, for evacuating air from the chest cavity, or pointing down, for evacuating fluid from the chest cavity between the chest wall and the lung. Also, such butterfly attachments are not suitable for large tube diameters required of chest drains, typically from 5 to 20 mm in diameter. Accordingly, although fixed orientation attachments such as butterfly attachments have been well known for use with small bore tubes such as central lines for very many years, it is also well known that they are not suitable for use for chest drains.

In light of these problems with known chest drains, there is a need for a simple, quick, secure and more comfortable chest drain securing system.

The present invention aims to meet this need and to overcome at least some of the problems of known chest drains, with their accompanying insertion and securing techniques, discussed hereinabove.

The present invention also aims to provide an improved drain tube assembly that may be utilised other than as a chest drain.

Accordingly, the present invention provides a drain tube assembly for draining a body cavity, the assembly comprising a flexible drain tube having a distal end for insertion into the body cavity and a proximal end, the distal end having at least one drainage hole therein, and at least one attachment ring so as to provide an attachment for suture, the attachment ring being connected to the drain tube by a flexible arm permitting rotational movement of the attachment ring relative to the drain tube.

The present invention further provides a kit of parts for a drain tube assembly for draining a body cavity, the kit of parts comprising a flexible drain tube having a distal end for insertion into the body cavity and a proximal end, the distal end having at least one drainage hole therein, the external surface of the drain tube having a radially outwardly directed ridge formed therein at least at one location between the distal and proximal ends of the drain tube, and an anchor piece adapted to be fitted around the drain tube to a mounting point located on the proximal side of and adjacent to the ridge whereby the ridge acts as a stop member preventing movement of the drain tube relative to the anchor piece in a proximal direction, the anchor piece comprising a collar, at least one attachment ring so as to provide an attachment for suture, and a flexible arm connecting the attachment ring to the collar, the flexible arm permitting rotational movement of the attachment ring relative to the collar.

The present invention further provides a drain tube assembly for draining a body cavity, the assembly comprising a flexible drain tube having a distal end for insertion into the body cavity and a proximal end, the distal end having at least one drainage hole therein, and at least one attachment for suture integral with a collar of an anchor piece that is disposed around the drain tube, the collar being tightly fitted around the drain tube at a selected longitudinal position along the drain tube.

The present invention further provides a kit of parts for a drain tube assembly for draining a body cavity, the kit of parts comprising a flexible drain tube having a distal end for insertion into the body cavity and a proximal end, the distal end having at least one drainage hole therein, and at least one attachment for suture integral with a collar of an anchor piece that is adapted to be disposed around the drain tube, the collar being adapted to be tightly fitted around the drain tube at a selected longitudinal position along the drain tube.

The present invention further provides an anchoring device for a drain tube assembly for draining a body cavity, the anchoring device comprising a collar adapted to be fitted around a drain tube, the collar comprising a locking clip having a central part and two opposed end parts, and the two end parts having complementary interlocking elements which are adapted to be selectively interlocked, to close the locking clip for securing the central part around the drain tube, and unlocked to open the locking clip, and at least one attachment ring so as to provide an attachment for suture, the attachment ring being flexibly connected to the collar thereby to permit movement of the attachment ring relative to the collar.

The present invention also provides a method of inserting a chest drain, the method comprising the steps of: providing a chest drain assembly comprising a flexible drain tube having a distal end for insertion into the body cavity and a proximal end, the distal end having at least one drainage hole therein, and at least one attachment ring so as to provide an attachment for suture, the attachment ring being connected to the drain tube by a flexible arm permitting rotational movement of the attachment ring relative to the drain tube; inserting the distal end into the chest cavity of a patient through a wound incision in the chest wall so that the at least one attachment ring is against the skin of the patient and so that the drain tube is at the desired insertion length and orientation; and suturing the at least one attachment ring to the skin.

The present invention further provides a method of inserting a chest drain, the method comprising the steps of: providing a chest drain assembly comprising a flexible drain tube having a distal end for insertion into the body cavity and a proximal end, the distal end having at least one drainage hole therein, and at least one attachment for suture integral with a collar of an anchor piece that is disposed around the drain tube; inserting the distal end into the chest cavity of a patient through a wound incision in the chest wall; tightly fitting the collar around the drain tube at a selected longitudinal position along the drain tube so that the at least one attachment for suture is against the skin of the patient and so that the drain tube is at the desired insertion length and orientation; and suturing the at least one attachment for suture to the skin.

The present invention yet further provides a method of inserting a chest drain, the method comprising the steps of: providing a flexible drain tube having a distal end for insertion into the body cavity and a proximal end, the distal end having at least one drainage hole therein, the external surface of the drain tube having an outwardly directed ridge formed therein at least at one location between the distal and proximal ends of the drain tube; inserting the distal end into the chest cavity of a patient so that the drain tube is at the desired insertion length and orientation and the ridge is proximal to the wound incision; fitting an anchor piece around the drain tube to a mounting point located on the proximal side of the ridge, the anchor piece comprising a collar for fitting around the drain tube, at least one attachment ring so as to provide an attachment for suture, and a flexible arm connecting the attachment ring to the collar, the flexible arm permitting rotational movement of the attachment ring relative to the collar; disposing the at least one attachment ring against the skin of the patient and the ridge just inside the wound incision; and suturing the at least one attachment ring to the skin.

Embodiments of the present invention will now be described, by way of example only, with reference to the accompanying drawings in which:

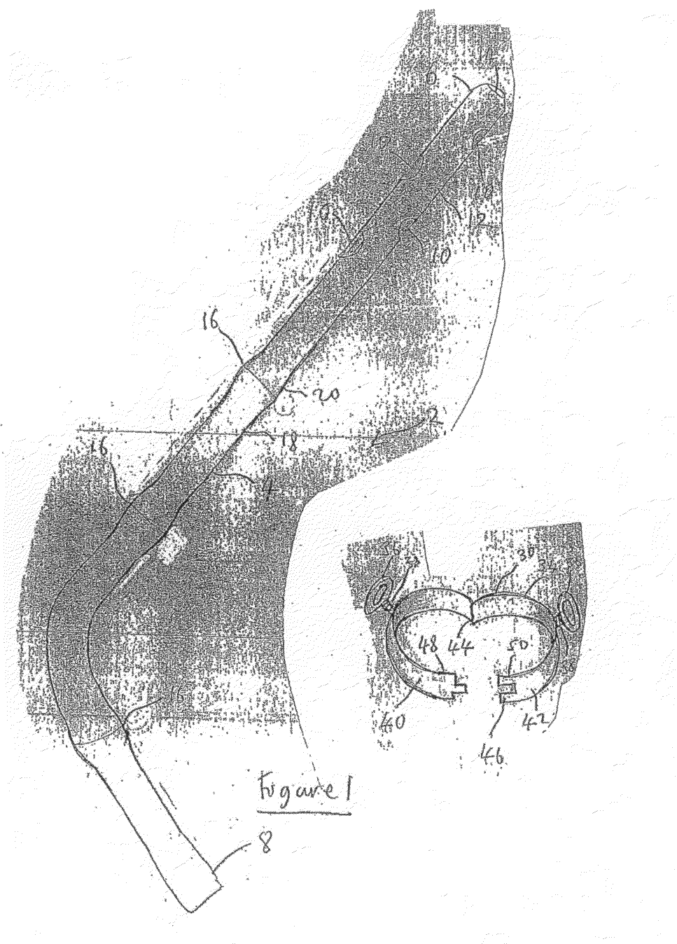

FIG. 1 is a schematic side elevation of a chest drain in accordance with a first embodiment of the present invention in a disassembled configuration;

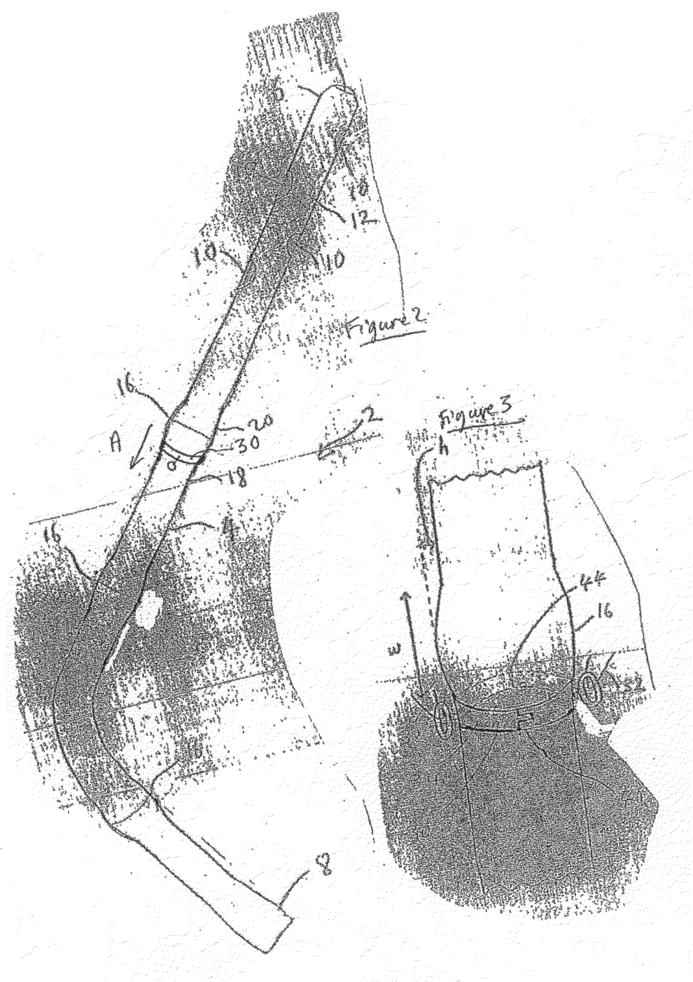

FIG. 2 is a schematic side elevation of the chest drain of FIG. 1 in an assembled configuration;

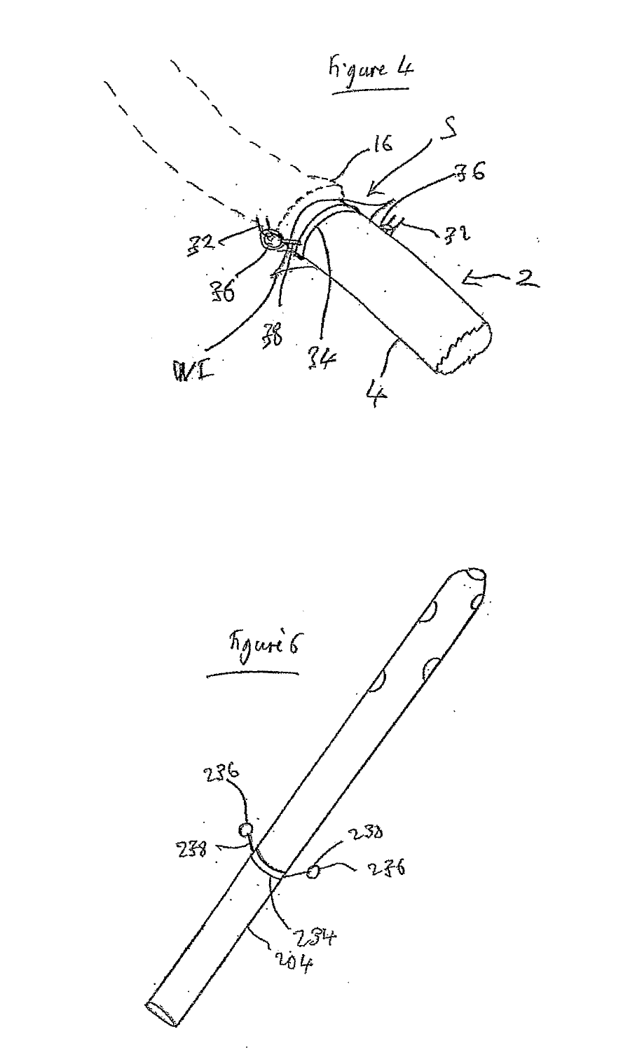

FIG. 3 is an enlarged schematic side elevation, partly in phantom, of a portion of the chest drain of FIG. 2 with the anchor piece fitted to the chest tube;

FIG. 4 is a schematic side elevation, partly in phantom, of the chest drain of FIG. 2 inserted into the chest cavity of a patient and secured to the skin of a patient by the anchor piece;

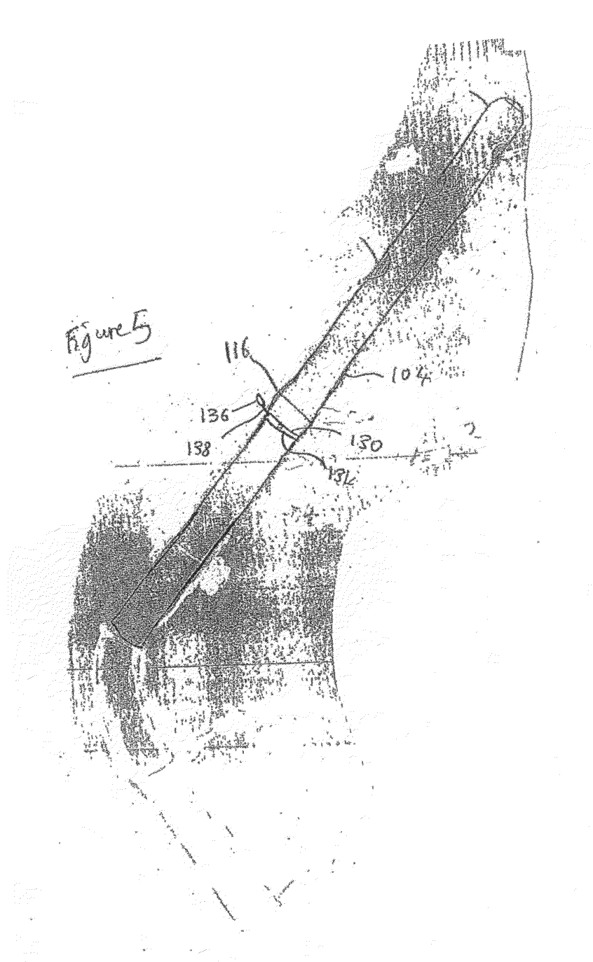

FIG. 5 is a schematic side elevation of a chest drain in accordance with a second embodiment of the present invention;

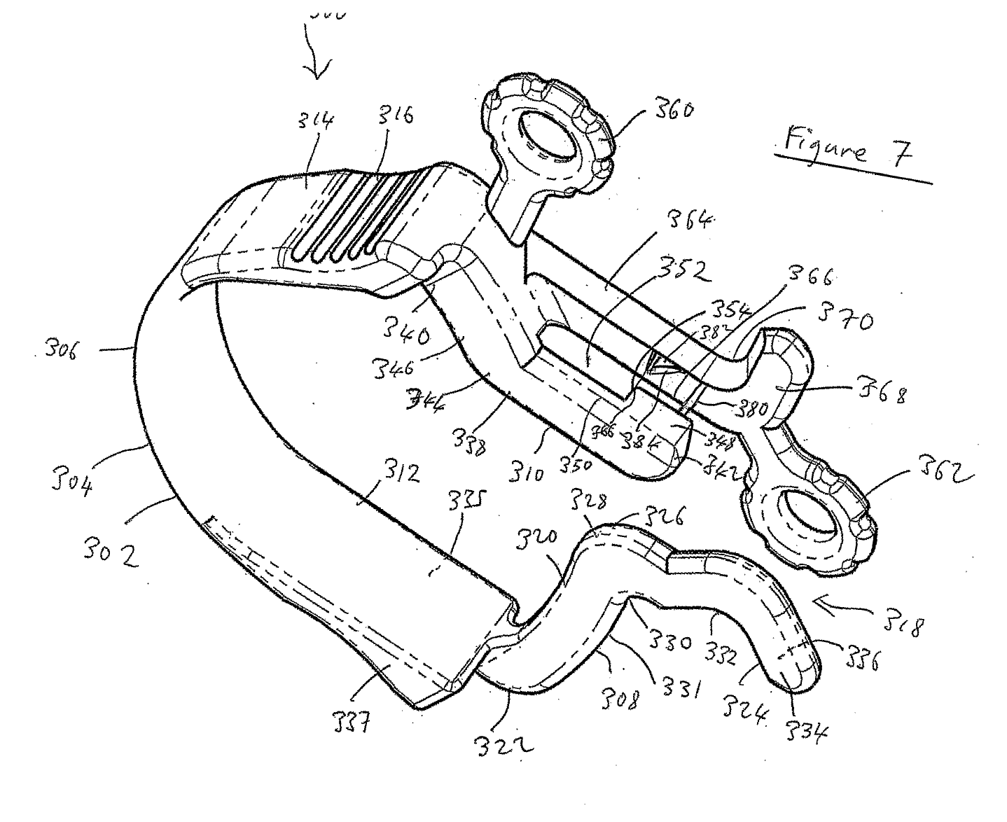

FIG. 6 is a schematic side elevation of a chest drain in accordance with a third embodiment of the present invention;

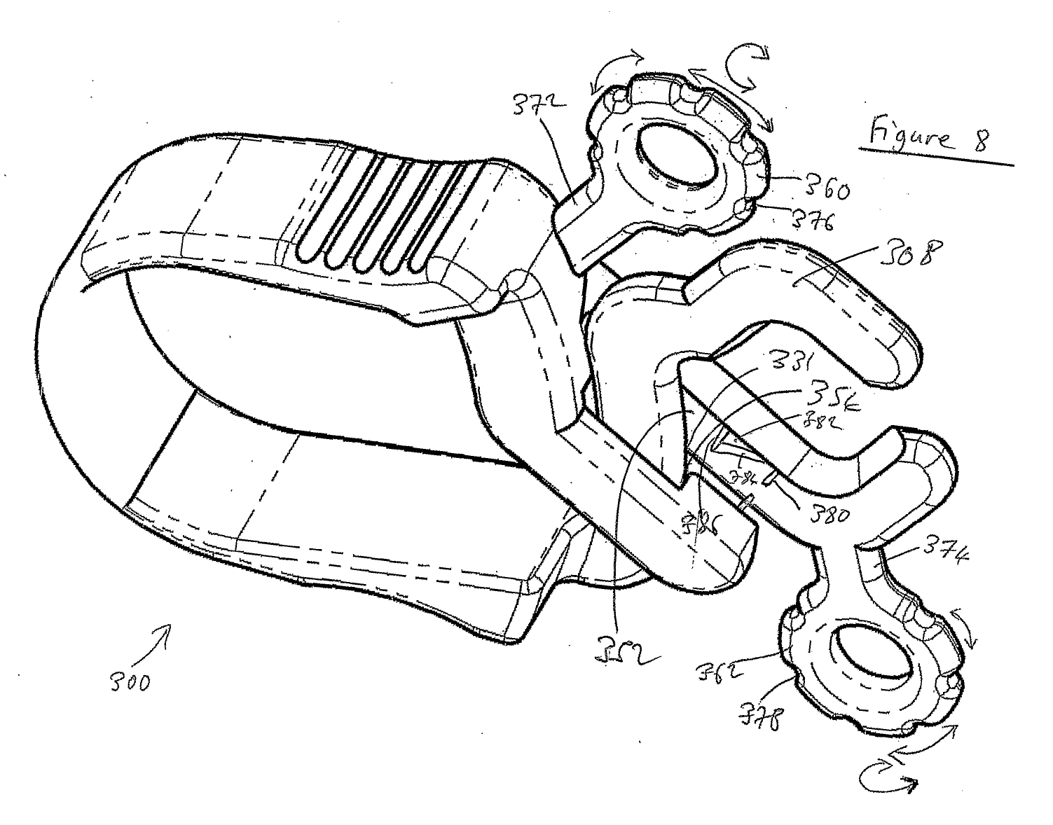

FIG. 7 is a schematic side perspective view of an anchoring device for a chest drain in accordance with a fourth embodiment of the present invention, the anchoring device being in an open unlocked configuration;

FIG. 8 is a schematic side perspective view of the anchoring device of FIG. 7, the anchoring device being in a closed locked configuration.

Referring to FIGS. 1 to 4, there is shown a chest drain in accordance with a first embodiment of the present invention. The chest drain, designated generally as (2), comprises a flexible chest tube (4) which is an elongate generally cylindrical hollow tubular member having a distal end (6) for insertion into the chest cavity of a patient and a proximal end (8) for connection to a drainage system (not shown) for collecting fluid drained by the chest drain. The tube (4) is typically made of medical grade plastic or rubber. The tube (4) has a constant internal diameter so as to provide an internal bore of constant width, which would reduce or obviate the likelihood of tube blockage. The tube wall has a thickness typical of known chest drain tubes. The tube (4) in this embodiment has a bend (5) preformed therein, but the tube (4) may be straight. At the distal end (6) a succession of drainage holes (10) are provided in the cylindrical wall (12) of the chest tube (4) at different radial locations, extending along a portion of the longitudinal length of the distal end (6) of the chest tube, and an end hole (14) is also provided at the distal end (6). These holes (10, 14) are provided for permitting fluid to enter the chest tube (4) from the chest cavity of the patient.

A radially outwardly directed ridge (16) formed in the exterior cylindrical surface (18) of the chest tube (4) is provided at least at one location between the distal and proximal ends (6, 8) of the chest tube (4). The ridge (16) extends circumferentially around the chest tube (4). The exterior circumferential surface (20) of the ridge (16) is smoothly contoured in a longitudinal direction of the tube so as to constitute a smooth low profile bulge in the tube side wall, and preferably has a symmetric section about a central transverse plane. Typically, the ridge (16) has a height (h) of from 3 to 5 mm and a longitudinal width (w) of from 7 to 15 mm depending on the size of the chest drain, typically providing a height/width aspect ratio of from 0.2 to 0.75, more particularly from 0.25 to 0.5, still more particularly from ⅓ to 3/7. As shown in FIG. 3, the height (h) is the transverse distance, orthogonal to the tube axis, from the top of the ridge to an extension of the outer surface of the tube where there is no ridge, and the longitudinal width (w) is the distance along the tube length from one edge of the ridge to the opposite edge of the ridge. Such smooth contouring of the ridge (16) on the external surface of the chest tube (4) provides that when the ridge (16) is inserted into the chest cavity of a patient, the external surface of the portion of the chest tube (4) received within the patient is sufficiently smoothly contoured so as to not to cause additional discomfort to the patient.

The chest drain (2) also comprises an anchor piece (30). In this embodiment, the anchor piece (30) is supplied separate from the chest tube (4), as shown in FIG. 1, and for clarity of illustration is shown enlarged relative to the tube. In use, the anchor piece (30) is fitted around the chest tube (4) at a selected longitudinal position, as shown in Figure 2. The anchor piece (30) has a tight or interference fit to the chest tube (4), and so secures the chest tube (4) to the chest wall of the patient at the selected longitudinal position. The anchor piece (30) is secured to the skin and subcutaneous tissue of the patient by suturing (32), as shown in FIG. 4.

Most preferably, at least two such ridges (16) are provided. In the illustrated embodiment, three ridges (16) are provided in a longitudinally spaced configuration along the chest tube (4). Plural ridges (16) are provided in this way so as to provide plural mounting points for the anchor piece (30), in turn providing plural choices of intrathoracic drain length and permitting accommodation for patients with large subcutaneous tissue thickness.

The anchor piece (30) is typically made of medical grade plastic or rubber, and preferably comprises a single integral moulding.

The anchor piece (30) comprises a collar (34). The collar (34) is preferably substantially rigid. The collar (34) has an internal diameter which is substantially the same as, or slightly smaller than, the external diameter of the chest tube (4) at locations between the ridges (16). Accordingly, when fitted to the chest tube (4) at a position slightly on the proximal side of a ridge (16), as shown in detail in FIG. 3, any inadvertent relative sliding movement, in a proximal direction, of the chest tube (4) relative to the anchor piece (30), as shown by arrow A, is inhibited by abutment of the collar (34) and the ridge (16). This abutment tends to prevent any sliding movement of the chest tube (4) relative to the anchor piece (30) in the direction of arrow A. Therefore the ridge (16) acts as a stop member preventing movement of the anchor piece (30) along the tube (4) in one direction, while being sufficiently smooth in profile that it can be inserted into the wound cavity without discomfort to the patient.

The collar (34) has at least one attachment ring (36) outwardly extending therefrom. In the illustrated embodiment, two attachment rings (36) are provided, preferably diametrically opposite each other when the collar (34) is closed around the tube (4). The attachment ring (36) defines a closed loop, typically circular or elliptical in shape, so as to provide an attachment for suture. The attachment ring (36) is connected to the collar (34) by a flexible arm (38). Each attachment ring (36) and associated arm (38) is a planar configuration that can be placed flat against the skin of the patient, and the two attachment rings (36) are co-planar. The flexible arm (38) permits the attachment ring (36) to rotate relative to the collar (34). Preferably, the rotational movement comprises one or both of a pivoting or rotational movement about an axis extending along the arm (38) caused by twisting the arm (38) and a flexing movement caused by bending the arm (38) along its length. This provides a highly flexible connection between on the one hand the attachment ring or rings (36) for the suture (32) and on the other hand the collar (34) and correspondingly the portion of the chest tube (4) fitted within the collar (34).

In this embodiment, the anchor piece (30) is originally separate from the chest tube (4) and is fitted to the chest tube (4) by the medical operative at the desired longitudinal position. In this embodiment, the collar (34) of the anchor piece (30) is openable and comprises two collar halves (40, 42), which are selectively moveable between an open position (FIG. 1) and a closed position (FIGS. 2 and 3). The two collar halves (40, 42) are hinged together at a common linear hinge (44) and an interlocking clip (46) is provided at the opposite free ends (48, 50) of the respective collar halves (40, 42). The opposite free ends (48, 50) typically constitute male and female interlock elements of the interlocking clip (46). Each collar half (40, 42) has a respective attachment ring (36) and corresponding arm (38) affixed thereto. In the open configuration, the open collar (34) can be fitted around the chest tube (4) at the selected longitudinal position and then the collar (34) can be closed so that the two interlock elements fit together in a secure manner and tightly fit the collar (34) around the chest tube (4) at the desired longitudinal position. When the anchor piece (30) is fitted to the tube (4), there is provided a tight fitting between the internal diameter of the anchor piece (30) and the external diameter of the tube (4).

As is well known to a person skilled in the art, chest tubes for chest drains have different bores or diameters with corresponding different lengths depending on the particular clinical requirements of the patient. Typical chest tubes have a diameter from about 0.5 cm to about 2 cm, with typically there being four to five length sizes for a given selected diameter. As is known to a person skilled in the art, the diameter of chest tubes is identified using “French sizes”. The chest tube (4) may be of small, medium or large bore, as well known to a person skilled in the art. Typically, small bore chest tubes have French size 10 to 14, with medium and large bore test tubes having larger diameters. Typically, medium bore drains of French size 16 and above have a length of from 40 to 51 cm. For such a size of the chest tube (4), typically the distance from the most proximal hole (12) on the distal end (6) and the first (most distal) ridge (16) would typically be about 13 cm and successive ridges (16) would be approximately 4 cm apart. However, these distances may be varied, depending on the clinical application.

The use of the chest drain in accordance with the present invention will now be described.

The distal end (6) of the chest tube (4) is inserted into the chest cavity of a patient through a wound incision WI in the chest wall until the distal end of the chest tube is at the desired depth and orientation in the chest cavity. The ridge or ridges (16) distal of the anchor piece (30) is or are inserted so as to be within the body of the patient. The smooth exterior surface profile of the ridges (16) permits ready insertion in this way without causing excessive additional discomfort to the patient. Then the collar (34) of the anchor piece (30) is securely fitted by clipping around the chest tube (4) at a location on the proximal side of a selected ridge (16) on the chest tube (4) which is located just within the body. Each attachment ring (36) and associated arm (38) can be placed flat against the skin S of the patient. This provides a firm grounding of the attachment rings (36) against the skin. Then sutures (32) are stitched so as to attach the attachment rings (36) to the skin and subcutaneous tissue of the patient. The inserted chest drain (2) is shown in FIG. 4. The wound incision is covered by wound dressing material held in place by self-adhesive dressing tape (not shown).

The angle between the skin and the tube (4) can be varied by twisting or pivoting of the arms (38) and the provision of the sutures (32) between the attachment rings (36) and the skin permit flexibility in the connection between the body and the chest drain. The provision of the flexible arms (38) permits the selected tube insertion depth and orientation to be securely maintained substantially irrespective of movement of the body of the patient, because such movement of the body of the patient would merely cause twisting of the attachment rings (36) on the arms (38), without causing corresponding movement of the chest tube portion inserted into the body of the patient. The smoothly curved profile of the ridges (16) permits any ridge (16) easily to be inserted into the body of a patient without excessive discomfort, and subsequently can be easily removed from the body when pulled out after use of the chest drain. However, the smoothly curved profile of the ridge (16) also prevents, by interference of the collar (34) of the anchor piece (30) with the associated ridge (16), the tendency of the chest drain accidentally to be pulled out of the body, either as a result of the weight of the tube, the weight of the connection to the drainage system, or because of inadvertent pulling of the external portion of the chest tube. The avoidance of accidental removal of the chest drain prevents the difficulty and discomfort, both for patient and doctor, of having to go through reinsertion or adjustment of the chest drain position and removal and reapplication of self-adhesive tape.

The present invention provides a chest drain which can be securely fitted to the body of a patient in a variety of different configurations. Therefore a single chest drain and securing means may be employed for a variety of different insertion depths. The use of a flexible interconnection between the chest drain and the body of a patient not only provides a secure fitting of the drain, but also is less uncomfortable for the patient and is easier and quicker for medical personnel to insert and remove the drain. The risk of infection also seems to be lowered by the flexible connection of the present invention. This can tend to reduce the length of hospital stay required by a patient.

Accordingly, the present invention provides a comfortable and convenient chest drain which may readily be secured to the body of a patient. No special training is required for securing the chest drain after insertion. The chest drain and the associated insertion technique may be applied to a variety of makes, models and sizes of chest drain. The chest drain provides a greater freedom of movement to the patient, for example to attend physiotherapy clinics, which may assist early patient recovery. The avoidance of the need to have large amounts of self-adhesive tape for securing the chest drain permits early recognition of wound infection.

In a second embodiment, as shown in FIG. 5, a single attachment ring (136), with an associated arm (138), is provided on a collar (134) of anchor piece (130) and the tube (104) is provided with a single ridge (116). The tube of the first embodiment may be used with the anchor piece of the second embodiment, and the anchor piece of the first embodiment may be used with the tube of the second embodiment.

In a third embodiment, as shown in FIG. 6, the attachment rings (236), with their associated arms (238), may be provided on a collar (234) integrally moulded with, or affixed to, the tube (204) at a predetermined longitudinal position. The tube (204) does not have any ridges as required by the first and second embodiments.

In a fourth embodiment of the invention, as shown in FIGS. 7 and 8, there is provided an anchoring device (300) for a drain tube assembly (not shown) for draining a body cavity. The anchoring device (300) comprises a collar (302) adapted to be fitted around the drain tube (not shown). The collar (302) comprises a locking clip (304) having a central part (306) and two opposed end parts (308, 310). The central part (306) is arcuate, and in particular is generally U-shaped, and has an inner concave surface (312) for fitting around the outer convex surface of a drain tube. The inner concave surface (312) is substantially flat, in the axial direction, thereby to provide a large surface area for frictionally engaging the outer convex surface of the drain tube, and reliably securing the locking clip (304) at a desired longitudinal position along the length of the drain tube. The outer convex surface (314) of the central part (306) has knurls or ridges (316) for manual engagement by a medical operative when fitting the anchoring device (300) to a drain tube. The knurls or ridges (316) prevent inadvertent slippage of the fingers and thumb of the medical operative relative to the locking clip (304).

The two end parts (308, 310) comprise complementary interlocking elements which are adapted to be selectively interlocked, to close the locking clip (304) for securing the central part (306) around the drain tube, and unlocked to open the locking clip (304). The two end parts (308, 310) are integral with the central part (306), and the locking clip (304) is composed of a resilient plastics material, for example comprising an injection moulded thermoplastics material. The locking clip (304) is a self-biased spring clip as a result of the resilience of the plastics material.

In an unbiased position the locking clip (304) is in an open unlocked configuration as shown in FIG. 7. The two end parts (308, 310) are separated. This provides an opening (318) therebetween through which a drain tube can be pushed, optionally by separating the two end parts (308, 310) even further by pushing then apart against the spring bias, either for fitting the locking clip (304) to the drain tube to assemble a chest drain assembly or removing the locking clip (304) from the drain tube after use.

In a biased position the locking clip (304) is in a closed locked configuration as shown in FIG. 8. The two end parts (308, 310) are pushed together against the bias of the locking clip (304) and then interlocked by clipping together the two end parts (308, 310), the bias of the locking clip (304) holding the locking clip (304) in the closed locked configuration. This closes the central part (306) around the drain tube and secures the locking clip (304) to the drain tube at a desired location therealong.

The first end part (308) has a curved arm (320) comprising a shoulder (322) attached to the central part (306), a free end (324), and an intermediate elbow (326) therebetween. The elbow (326) has an outer convex surface (328) and an inner concave surface (330), the inner concave surface (330) defining a sharp corner, typically approximately a right angle. A first interlocking surface (331) on the first end part (308) is thereby provided between the inner concave surface (330) and the shoulder (322). The free end (324) includes a curved surface (332), adjacent to the inner concave surface (330), that is curved and adapted to be engageable by a finger or thumb of a user. The curved arm (320) has planar top and bottom surfaces (334, 336) that are parallel, and so the curved arm (320) is straight. The curved arm (320) is located in an offset position relative to the central part (306), being located nearer, in an axial direction, towards a bottom surface (335) than to a top surface (337) of the central part (306).

The second end part (310) has a curved arm (338) comprising a shoulder (340) attached to the central part (306), a free end (342), and an intermediate elbow (344) therebetween.

The elbow (344) has an obtuse angle. Both an upper arm portion (346) located between the shoulder (340) and the elbow (344) and an extremity (348) of the free end (342) are thicker in the axial direction than a lower arm portion (350) portion between the elbow (344) and the extremity (348). This forms a notch (352) adjacent the lower arm portion (350), the notch (352) being between the upper arm portion (346) and the thickened extremity (348). The notch (352) and the thickened extremity (348) of the free end (342) are aligned, in an axial direction, and preferably coplanar, with the curved arm (320) of the first end part (308). The edge of the thickened extremity (348) facing the notch (352) comprises a second interlocking surface (354) on the second end part (310).

When a user wishes to clamp the locking clip (304) onto the drain tube, so as to be in the closed locked configuration as shown in FIG. 8, the free ends (324, 342) of the first and second end parts (308, 310) are manually squeezed together against the bias of the sprung locking clip (304). The free end (324) of the first end part (308) is twisted in a first axial direction relative to the free end (342) of the second end part (310), causing the first end part (308) to be slid over and past the thickened extremity (348) and, on release, to be captured in the notch (352) by a clipping action. The first and second end parts (308, 310) are thereby interlocked by fitting the curved arm (320) into the notch (352) so that the first and second interlocking surfaces (330, 354) abut. The locking clip (304) is prevented from inadvertent opening by urging, as a result of the spring bias of the first interlocking surface (330) on the first end part (308) against the second interlocking surface (354) on the second end part (310). The first interlocking surface (330) extends over a distance and so the diameter of the central part (306) can vary in the interlocked configuration, thereby accommodating a range of diameters of the drain tube that can be clipped by the locking clip (304).

After use, the locking clip (304) can be manually released by the reverse operation, namely by twisting the free end (324) of the first end part (308) in an opposite second axial direction relative to the free end (342) of the second end part (310). The spring bias then causes the first end part (308) to be slid backwards over and past the thickened extremity (348) to open the locking clip (304). The drain tube can then be released, and removed through the opening (318).

In this embodiment two attachment rings (360, 362) are integral with the locking clip (304) so as to provide two attachments for suture. A first attachment ring (360) is provided on the shoulder (340) of the second end part (310). A second attachment ring (362) is provided at the end of an extension member (364) extending from the shoulder (340) of the second end part (310). The extension member (364) is generally aligned with the lower arm portion (350) and the thickened extremity (348), thereby forming a channel (366) between the thickened extremity (348) and the extension member (364). The first end member (308) is passed through the channel (366) when locking and unlocking the locking clip (304). The extension member (364) has a free end (368) that includes a curved concave surface (370) that is adapted to be engageable by a finger or thumb of a user. Therefore the locking clip (304) can be closed by manual engagement of both the curved concave surface (332) and the curved concave surface (370) on the first and second end parts (308, 310) respectively.

Each attachment ring (360, 362) is flexibly connected to the locking clip (304) thereby to permit rotational movement of the attachment ring (360, 362) relative to the locking clip (304) about any of three mutually orthogonal axes, as shown by the arrows in FIG. 8. The attachment ring (360) is connected to the shoulder (340) by an associated flexible integral arm (372). The attachment ring (362) is connected to the extension member (364) by an associated flexible integral arm (374).

The outer annular surface of each attachment ring (360, 362) is provided with a series of angularly spaced indentations (376, 378). The indentations (376, 378) are shaped and dimensioned to hold suture sewn through and around the respective attachment ring (360, 362) in a fixed angular position, This assists the locking clip (304) being securely sewn onto the patient's skin in the desired position without inadvertently working loose over time.

Typically, the locking clip (304) would be provided to the medical personnel in a sterilised package, optionally packaged together with an associated drain tube. The locking clip (304) would most preferably be disposable, although it could be reusable provided it could be readily sterilised. For a disposable locking clip (304), there is a desire to prevent inadvertent or deliberate reuse, in order to reduce the possibility of infection. In this embodiment, means are provided to indicate whether or not the locking clip (304) has been used, and means are also provided to inhibit the locking clip (304) from being reused. In other embodiments only one or none of these means may be provided.

A frangible tear strip (380) extends across the channel (366). When the first end member (308) is passed through the channel (366) to lock the locking clip (304), the frangible tear strip (380) is broken, and this indicates that the locking clip (304) has been used.

A one-way abutment (382) having a ramp surface (384) and a stop surface (386) is also provided in the channel (366). When the first end member (308) is passed through the channel (366) to lock the locking clip (304), the first end member (308) slides up and over the ramp surface (384) and snaps down, as a relaxation fit, past stop surface (386) into the notch (352). The stop surface (386) of the one-way abutment (382) inhibits or prevents removal of the first end member (308) from the notch (352) when in the interlocked position, and so inhibits or prevents the locking clip (304) from being reused.

Although the drain tube assembly has been disclosed with particular reference to a chest drain, the drain tube assembly of the present invention may be used for other drain tubes for draining other body cavities that require variable length catheters.

1. A drain tube assembly for draining a body cavity, the assembly comprising a flexible drain tube having a distal end for insertion into the body cavity and a proximal end, the distal end having at least one drainage hole therein, and at least one attachment ring so as to provide an attachment for suture, the attachment ring being connected to the drain tube by a flexible arm permitting rotational movement of the attachment ring relative to the drain tube.

2. A drain tube assembly according to claim 1 comprising two attachment rings located on opposed sides of the drain tube.

3. A drain tube assembly according to claim 1 wherein the at least one attachment ring and associated arm are integral with a collar of an anchor piece that is disposed around the drain tube.

4. A drain tube assembly according to claim 3 wherein the anchor piece is clipped to the drain tube in a selected position.

5. A drain tube assembly according to claim 4 wherein the collar of the anchor piece comprises two collar parts which have been clipped together in a closed configuration around the drain tube.

6. A drain tube assembly according to claim 5 wherein the two collar parts are hinged together at a common hinge and an interlocking clip is provided at the opposite free ends of the respective collar parts.

7. A drain tube assembly according to claim 5 wherein each collar part has a respective attachment ring and associated arm thereon.

8. A drain tube assembly according to claim 4 wherein the internal diameter of the collar of the anchor piece is substantially the same as, or slightly smaller than, the external diameter of the drain tube so that when the anchor piece is clipped to the tube there is provided a tight non-sliding fitting between the anchor piece and the drain tube.

9. A drain tube assembly according to claim 4 wherein the external surface of the drain tube has a radially outwardly directed ridge formed therein at least at one location between the distal and proximal ends of the drain tube, and the anchor piece is fitted to a mounting point on the drain tube located on the proximal side of and adjacent to the ridge whereby the ridge acts as a stop member preventing movement of the drain tube relative to the anchor piece in a proximal direction.

10. A drain tube assembly according to claim 9 wherein the ridge extends circumferentially around the drain tube.

11. A drain tube assembly according to claim 9 wherein the exterior surface of the ridge is smoothly contoured in a longitudinal direction of the drain tube so as to constitute a smooth low profile bulge in the drain tube.

12. A drain tube assembly according to claim 11 wherein the ridge has a symmetric section about a central transverse plane.

13. A drain tube assembly according to claim 9 wherein the ridge has a height (h) of from 3 to 5 mm and a longitudinal width (w) of from 7 to 15 mm.

14. A drain tube assembly according to claim 9 wherein the ridge has a height (h)/width (w) aspect ratio of from ⅓ to 3/7.

15. A drain tube assembly according to claim 9 wherein at least two ridges are provided in a longitudinally spaced configuration along the drain tube so as to provide plural mounting points for selectively fitting the anchor piece.

16. A drain tube assembly according to claim 3 wherein the anchor piece is integrally moulded with, or affixed to, the drain tube at a predetermined longitudinal position.

17. A drain tube assembly according to claim 1 which is a chest drain.

18. A kit of parts for a drain tube assembly for draining a body cavity, the kit of parts comprising a flexible drain tube having a distal end for insertion into the body cavity and a proximal end, the distal end having at least one drainage hole therein, the external surface of the drain tube having a radially outwardly directed ridge formed therein at least at one location between the distal and proximal ends of the drain tube, and an anchor piece adapted to be fitted around the drain tube to a mounting point located on the proximal side of and adjacent to the ridge whereby the ridge acts as a stop member preventing movement of the drain tube relative to the anchor piece in a proximal direction, the anchor piece comprising a collar, at least one attachment ring so as to provide an attachment for suture, and a flexible arm connecting the attachment ring to the collar, the flexible arm permitting rotational movement of the attachment ring relative to the collar.

19. A kit of parts for a drain tube assembly according to claim 18 comprising two attachment rings located on opposed sides of the collar.

20. A kit of parts for a drain tube assembly according to claim 18 wherein the at least one attachment ring and associated arm are integral with the collar.

21. A kit of parts for a drain tube assembly according to claim 18 wherein the anchor piece is adapted to be clipped to the drain tube in a selected position.

22. A kit of parts for a drain tube assembly according to claim 21 wherein the collar of the anchor piece comprises two collar parts which are adapted to be clipped together in a closed configuration around the drain tube.

23. A kit of parts for a drain tube assembly according to claim 22 wherein the two collar parts are hinged together at a common hinge and an interlocking clip is provided at the opposite free ends of the respective collar parts.

24. A kit of parts for a drain tube assembly according to claim 22 wherein each collar part has a respective attachment ring and associated arm thereon.

25. A kit of parts for a drain tube assembly according to claim 18 wherein the internal diameter of the collar is substantially the same as, or slightly smaller than, the external diameter of the drain tube so that when the anchor piece is clipped to the tube there is provided a tight non-sliding fitting between the anchor piece and the drain tube.

26. A kit of parts for a drain tube assembly according to claim 18 wherein the ridge extends circumferentially around the drain tube.

27. A kit of parts for a drain tube assembly according to claim 18 wherein the exterior surface of the ridge is smoothly contoured in a longitudinal direction of the drain tube so as to constitute a smooth low profile bulge in the drain tube.

28. A kit of parts for a drain tube assembly according to claim 18 wherein the ridge has a symmetric section about a central transverse plane.

29. A kit of parts for a drain tube assembly according to claim 18 the ridge has a height (h) of from 3 to 5 mm and a longitudinal width (w) of from 7 to 15 mm.

30. A kit of parts for a drain tube assembly according to claim 18 wherein the ridge has a height (h)/width(w) aspect ratio of from ⅓ to 3/7.

31. A kit of parts for a drain tube assembly according to claim 18 wherein at least two ridges are provided in a longitudinally spaced configuration along the drain tube so as to provide plural mounting points for selectively fitting the anchor piece.

32. A kit of parts for a drain tube assembly according to claim 18 which is a chest drain.

33. A drain tube assembly for draining a body cavity, the assembly comprising a flexible drain tube having a distal end for insertion into the body cavity and a proximal end, the distal end having at least one drainage hole therein, and at least one attachment for suture integral with a collar of an anchor piece that is disposed around the drain tube, the collar being tightly fitted around the drain tube at a selected longitudinal position along the drain tube.

34. (canceled)

35. (canceled)

36. (canceled)

37. (canceled)

38. (canceled)

39. (canceled)

40. A kit of parts for a drain tube assembly for draining a body cavity, the kit of parts comprising a flexible drain tube having a distal end for insertion into the body cavity and a proximal end, the distal end having at least one drainage hole therein, and at least one attachment for suture integral with a collar of an anchor piece that is adapted to be disposed around the drain tube, the collar being adapted to be tightly fitted around the drain tube at a selected longitudinal position along the drain tube.

41. (canceled)

42. (canceled)

43. (canceled)

44. (canceled)

45. (canceled)

46. An anchoring device for a drain tube assembly for draining a body cavity, the anchoring device comprising a collar adapted to be fitted around a drain tube, the collar comprising a locking clip having a central part and two opposed end parts, and the two end parts having complementary interlocking elements which are adapted to be selectively interlocked, to close the locking clip for securing the central part around the drain tube, and unlocked to open the locking clip, and at least one attachment ring so as to provide an attachment for suture, the attachment ring being flexibly connected to the collar thereby to permit movement of the attachment ring relative to the collar.

47. An anchoring device according to claim 46 wherein the central part is arcuate and has an inner concave surface for fitting around the outer convex surface of a drain tube.

48. An anchoring device according to claim 47 wherein the locking clip is composed of a resilient plastics material so as to comprise is a self-biased spring clip.

49. An anchoring device according to claim 48 wherein the two end parts are integral with the central part.

50. An anchoring device according to claim 48 wherein in an unbiased position the locking clip is in an open unlocked configuration and the two end parts are separated to provide an opening therebetween.

51. An anchoring device according to claim 48 wherein in a closed locked configuration the locking clip the two end parts are pushed together against the bias of the locking clip and interlocked by clipping together the two end parts, the bias of the locking clip holding the locking clip in the closed locked configuration.

52. An anchoring device according to claim 48 wherein the first end part has a first curved arm defining a first interlocking surface and the second end part has a second curved arm defining a second interlocking surface adjacent a notch, and the first and second ends are interlocked by fitting the first curved arm into the notch so that the first and second interlocking surfaces abut.

53. An anchoring device according to claim 52 wherein the first curved arm is straight and the notch is between a thickened the upper arm portion and a thickened extremity at a free end of the second curved arm.

54. An anchoring device according to claim 53 wherein the notch is aligned, in an axial direction, with the first curved arm.

55. An anchoring device according to claim 54 wherein the first curved arm is located in an offset position, in an axial direction, relative to the central part.

56. An anchoring device according to claim 46 wherein two mutually spaced attachment rings are provided integral with the locking clip.

57. An anchoring device according to claim 56 wherein the two attachment rings are provided on a second end part of the locking clip.

58. An anchoring device according to claim 57 wherein a first attachment ring is provided on a shoulder of the second end part, the shoulder being connected to the central part, and a second attachment ring is provided at the end of an extension member extending from the shoulder of the second end part.

59. An anchoring device according to claim 58 wherein the extension member is generally aligned with at least a lower portion of the second end part thereby forming a channel between the second end part and the extension member, the first end part being passable through the channel when locking and unlocking the locking clip.

60. An anchoring device according to claim 59 wherein the extension member has a free end that includes a curved concave surface that is adapted to be engageable by a finger or thumb of a user.

61. An anchoring device according to claim 60 wherein the first end parts has a curved concave surface that is adapted to be engageable by a finger or thumb of a user.

62. An anchoring device according to claim 46 wherein the at least one attachment ring is flexibly connected to the locking clip by an associated flexible integral arm.

63. An anchoring device according to claim 46 wherein the at least one attachment ring is provided on an outer circumferential surface thereof with a series of angularly spaced indentations which are shaped and dimensioned to hold suture sewn through and around the respective attachment ring in a fixed angular position,

64. An anchoring device according to claim 46 further comprising at least one of means to indicate whether or not the locking clip has been used, and means to inhibit the locking clip from being reused.

65. An anchoring device according to claim 64 wherein the means to indicate whether or not the locking clip has been used comprises a frangible tear strip which is broken when the first end part is moved to interlock with the second end part.

66. An anchoring device according to claim 64 wherein the means to inhibit the locking clip from being reused comprises a one-way abutment to retain the first end part in an interlocked position with the second end part, the first and second end parts being fitted into the interlocked position by a relaxation fit over the one-way abutment.

67. (canceled)

68. (canceled)

69. A method of inserting a chest drain, the method comprising the steps of: providing a chest drain assembly comprising a flexible drain tube having a distal end for insertion into the body cavity and a proximal end, the distal end having at least one drainage hole therein, and at least one attachment ring so as to provide an attachment for suture, the attachment ring being connected to the drain tube by a flexible arm permitting rotational movement of the attachment ring relative to the drain tube; inserting the distal end into the chest cavity of a patient through a wound incision in the chest wall so that the at least one attachment ring is against the skin of the patient and so that the drain tube is at the desired insertion length and orientation; and suturing the at least one attachment ring to the skin.

70. A method of inserting a chest drain, the method comprising the steps of: providing a chest drain assembly comprising a flexible drain tube having a distal end for insertion into the body cavity and a proximal end, the distal end having at least one drainage hole therein, and at least one attachment for suture integral with a collar of an anchor piece that is disposed around the drain tube; inserting the distal end into the chest cavity of a patient through a wound incision in the chest wall; tightly fitting the collar around the drain tube at a selected longitudinal position along the drain tube so that the at least one attachment for suture is against the skin of the patient and so that the drain tube is at the desired insertion length and orientation; and suturing the at least one attachment for suture to the skin.

71. A method of inserting a chest drain, the method comprising the steps of: providing a flexible drain tube having a distal end for insertion into the body cavity and a proximal end, the distal end having at least one drainage hole therein, the external surface of the drain tube having an outwardly directed ridge formed therein at least at one location between the distal and proximal ends of the drain tube; inserting the distal end into the chest cavity of a patient so that the drain tube is at the desired insertion length and orientation and the ridge is proximal to the wound incision; fitting an anchor piece around the drain tube to a mounting point located on the proximal side of the ridge, the anchor piece comprising a collar for fitting around the drain tube, at least one attachment ring so as to provide an attachment for suture, and a flexible arm connecting the attachment ring to the collar, the flexible arm permitting rotational movement of the attachment ring relative to the collar; disposing the at least one attachment ring against the skin of the patient and the ridge just inside the wound incision; and suturing the at least one attachment ring to the skin.
